(19) United States
(12) Patent Application Publication
(10) Pub. No.: US 2016/0000890 A1
(43) Pub. Date: Jan. 7, 2016

(54) THERMOSENSITIVE HYDROGEL COLLAGENASE FORMULATIONS

(71) Applicant: BioSpecifics Technologies Corp., Lynbrook, NY (US)

(72) Inventors: Bo Yu, Fresh Meadows, NY (US); Thomas L. Wegman, North Merrick, NY (US)

(73) Assignee: BIOSPECIFICS TECHNOLOGIES CORP., Lynbrook, NY (US)

(21) Appl. No.: 14/853,245

(22) Filed: Sep. 14, 2015

Related U.S. Application Data
(63) Continuation-in-part of application No. PCT/US2014/029448, filed on Mar. 14, 2014.
Application No. PCT/US2014/029448 is a continuation-in-part of application No. PCT/US2015/011296, filed on Jan. 14, 2015.
(60) Provisional application No. 61/790,070, filed on Mar. 15, 2013.
(60) Provisional application No. 62/063,056, filed on Oct. 13, 2014.

(51) Int. Cl. A61K 38/48; B65D 85/00; A61K 47/18; A61K 47/34; A61K 47/32

(52) U.S. Cl. CPC A61K 38/4886; A61K 47/34; A61K 47/32; A61K 47/18; B65D 85/70

(57) ABSTRACT

It is an object of the present disclosure to provide a formulation for injectable and topical collagenase, which will have extended residence time for the drug at the therapeutic targeted area for the indication being treated. It is a further object of the disclosure to provide a slow release formulation for collagenase, which is compatible with the active ingredient and does not adversely affect its activity. Still a further object of the disclosure is to provide an injectable formulation for collagenase which can be effectively administered to a patient with a small size needle without exhibiting pre-gelation, which would interfere with the ability to deliver the required dose for treatment. Still a further object of the disclosure is to provide a water-based topical formulation for collagenase which will be more compatible with other topically used medications to achieve better results.

### CROSS-REFERENCE TO RELATED APPLICATIONS

This application is a continuation-in-part of and claims priority of PCT/US2014/029448, filed Mar. 14, 2014, which claims priority of U.S. Provisional Application Ser. No. 61/790,070, filed Mar. 15, 2013, and of PCT/US2015/011296, filed Jan. 14, 2015, which claims priority of U.S. Provisional Application Ser. No. 62/063,056, filed Oct. 13, 2014, all of which are hereby incorporated by reference in their entireties.

### FIELD OF THE INVENTION

A sterile formulation for injectable and topical collagenase which will have extended residence time for the drug at the therapeutic targeted area for the indication being treated, methods of use of such formulation and processes for its preparation.

### BACKGROUND OF THE INVENTION

At present a collagenase consisting of a fixed-ratio mixture of Aux I and Aux II collagenases derived from Clostridium histolyticum has been approved for use as a prescription medicine in the United States under the trademark Xiaflex® and in the European Union under the trademark Xiapex®. Current approved indications are for the treatment of adults suffering from Dupuytren contraction and for adult men who have Peyronies disease. In addition, this product is under clinical and pre-clinical investigation for a number of collagen lesion based human and veterinary applications such as frozen shoulder, human lipoma, canine lipoma, cellulite, uterine fibroids, chronic dermal ulcers and severely burned areas.

All of the aforesaid non-topical applications require local (lesion site) injection of the collagenase product. It is highly desirable that to achieve optimum clinical benefit that the collagenase remain at the lesion site for an extended period to allow the enzyme to work to maximum extent. However, the current commercial formulation of collagenase for injection is a solution prepared by reconstituting the lyophilized collagenase powder with buffered saline for injection. Data from a pharmacokinetic study has shown that a significant amount of collagenase in the commercial formulation is found in patient urine as early as thirty minutes post injection. This indicates that the administered collagenase may be washed away easily from the injection site at the lesion or other therapeutic targeted area. It is logical that formulations that provide longer residence time at the injection site can improve the therapeutic effect of the collagenase treatment. As for the topical indications, current available topical collagenase is a petrolatum-based ointment. Water-based formulations are desirable but difficult to develop due to the fact that collagenases are not stable in water long term. Water-based formulations are much more amenable to changing or adding different medications.

### BRIEF DESCRIPTION OF THE INVENTION

It is an object of the present invention to provide a formulation for injectable collagenase, which will have extended residence time for the drug at the therapeutic targeted area for the indication being treated. It is a further object of the invention to provide a slow release formulation for collagenase, which is compatible with the active ingredient and does not adversely affect its activity. Still a further object of the invention is to provide an injectable formulation for collagenase which can be effectively administered to a patient with a small size needle without exhibiting pre-gelation, which would interfere with the ability to deliver the required dose for treatment.

As used herein the term “collagenase” is meant to include one or more proteins exhibiting collagenase activity in a standard collagenase assay, preferably an Aux I and/or an Aux II collagenase derived from histolyticum, most preferably a 1:1 mixture of such Aux I and Aux II collagenases.

It has now been found and forms the basis of the present invention that a compatible, injectable formulation for providing a slow release of collagenase at the therapeutic targeted site can be prepared using specific reverse thermogeling hydrogels. Such hydrogels are fluid at room temperature but form a gel at the higher interbody temperature, which gel can entrap substantial amounts of the collagenase at the injection site in the body for extended release at the desired location.

Thermogelling hydrogels for delivery of therapeutic drugs is still a fairly new technology and there are still many problems to solve to achieve the desired objects of this invention. One problem is the injectability or syringeability problem which represents a critical issue for clinical usage. See for example, T. R. Hoare and D. S. Kohane, Polymer's 49 (2008) 1993-2007. High viscosity and premature gelation inside the needle are the two aspects of such injectability problem. It is common that the polymers solution comprising the hydrogel is viscous at a room temperature of about 24° C. The “thick” solution is a complication for the clinician who is administering the solution through a syringe. In order to improve patient acceptance of procedures involving multiple injections it is highly desired to use a small size needle in the syringe. However, when the scientific literature is reviewed it is interesting to observe that when hydrogels have been reported to have been injected into animals numerous citations indicate the use of large size syringes and needles. For example, ReGe®, a triblock copolymer has been used to inject drugs in humans using a 23 G½ sized needle (Anti-cancer Drugs, 2007, vol 18, No 3).

Due to the thermoresponsive properties of the prior hydrogel compositions, gelation inside the needle can occur after penetration of the skin but prior to discharging the contents of the syringe thus plugging up the needle. Thus, in order to have acceptable injectability for a collagenase hydrogel formulation the formulation must demonstrate that: (1) the collagenase hydrogel solution can be handled comfortably with a 0.5 mL syringe fitted with a 28 G½ needle at room temperature; and (2) the needle will not exhibit pre-gelation after the needle has penetrated through the skin for a reasonable time—thus allowing the content of the syringe to be administered under normal conditions of treatment with collagenase for injection.

It is desired that the in situ gelation of the thermosensitive hydrogel/collagenase formulation at the therapeutic targeted site will entrap at least about 70 wt % of the amount of the collegenase originally contained in the original solution in the syringe and most preferably at least 80 wt % of such collagenase. The amount of collagenase in an injectable dose for present approved indications is about 0.58 mg, although the formulation can be adapted to contain more or less collagenase for other indications, which may be approved in the future. The non-entrapped portion of the administered collagenase is available for immediate treatment of the target collagen lesion while the entrapped collagenase will be released over a period of time to allow for extended treatment from the single injection. Unlike conventional gel formulations for extended release of systemic therapeutic drugs which can have release times of several weeks or even months, the release period for the collagenase gels should not exceed a few days, preferable about two days from the time of injection. Such a regime may reduce the number of injections needed for effective treatment of the lesion with minimum risk of undesired side effects from exposure of normal tissue to collagenase thus resulting in a high level of patient acceptance of this modality of treatment.

### DETAILED DESCRIPTION OF THE INVENTION

Collagenase for use according to the invention may be obtained from any convenient source, including mammalian (e.g., human, porcine), crustacean (e.g., crab, shrimp), fungal, and bacterial (e.g., from the fermentation of Clostridium, Streptomyces, Pseudomonas, Vibrio or Achromobacter iophagus). Collagenase can be isolated from a natural source or can be genetically engineered/recombinant. One common source of crude collagenase is from a bacterial fermentation process, specifically the fermentation of Clostridium histolyticum. The crude collagenase obtained from C. histolyticum can be purified using any of a number of techniques known in the art of protein purification, including chromatographic techniques. Collagenase compositions useful for the invention also can be prepared using any commercially available or isolated collagenase activity, or by mixing such activities. For example, purified collagenase can be provided by Biospecifics Technologies, Lynbrook, N.Y.

Preferred collagenases for use in the invention are from C. histolyticum, i.e., collagenase class I and class II. A practical advantage of using C. histolyticum for the production of collagenases is that it can be cultured in large quantities in simple liquid media, and it regularly produces amounts of proteolytic enzymes which are secreted into the culture medium. Bovine products have been used in culture media in the fermentation of C. histolyticum, but these run the risk of contamination by agents which cause transmissible spongiform encephalopathies (TSEs; e.g., prions associated with bovine spongiform encephalopathy or “mad cow disease”). Therefore, it is preferred to avoid such bovine products. An animal-product-free system is preferred. The H4 strain of Clostridium histolyticum, originally developed in 1956 can serve as a source for cells for culture. This strain, and a strain derived from the H4 strain, named the ABC Clostridium histolyticum master cell bank (deposited as ATCC 21000) were developed using animal products, but are suitable to use in the invention.

U.S. Pat. No. 7,811,560, incorporated herein by reference in its entirety for all purposes, discloses methods of producing collagenases, and is incorporated herein in its entirety for all purposes. Using soybean derived fermentation medium, the methods described therein generated separately highly purified collagenase I and II. This patent also discloses methods of producing highly purified collagenases using culture media containing porcine-derived products. Any of these methods are suitable for use with the invention. U.S. Patent Publication 2010/0086971, incorporated herein by reference in its entirety for all purposes, discloses numerous fermentation recipes which are based on vegetable peptone, including soybean-derived peptone, or vegetable-derived peptone plus fish gelatin. The methods described in this publication are suitable to produce growth of Clostridium and collagenase activities. These methods also are suitable and contemplated for use with the invention, however any method known in the art of producing collagenase enzyme activity may be used.

In preferred culture methods, the peptone is from a plant source selected from the group consisting of soy bean, broad bean, pea, potato, and a mixture thereof. The peptone may be selected from the group consisting of Oxoid VG100 Vegetable peptone No. 1 from pea (VG100), Oxoid VG200 Vegetable peptone phosphate broth from Pea (VG200), Merck TSB CASO-Bouillion animal-free (TSB), Invitrogen Soy bean peptone No 110 papainic digest (SP6), Fluka Broad bean peptone (BP), Organotechnie Plant peptone E1 from potato (E1P), BBL Phytone™ peptone and BD Difco Select Phytone™.

It is preferable that a single type of peptone is present in the nutrient composition, whereby the peptone is selected from the group consisting of BP, E1P, Soy bean peptone E110, VG100, and VG200, and whereby the concentration of the peptone in the composition is about 5% weight by volume. More preferably, a single type of peptone is present in the nutrient composition, whereby the peptone is BBL phytone peptone or Difco Select Phytone™ UF, and whereby the concentration of the peptone in the composition is about 10-13% weight by volume.

Preferred methods of isolating collagenase avoid undesirable contaminating proteases such as clostripain. Clostripain, a cysteine protease, is believed to be a major cause of collagenase degradation and instability, and is present in Clostridium culture. When such proteases are present in a crude collagenase mixture, one must take extra precautions to neutralize the proteases, including using protease inhibitors, such as leupeptin, and performing all of the purification steps in specially designed cold rooms with chilled solutions to reduce protease activity. Preferred methods of isolation therefore take advantage of one of two approaches to avoid clostripain: remove clostripain as early as possible in the purification method or reduce clostripain production during the fermentation stage.

Preferred collagenase compositions are produced by fermenting C. histolyticum in medium free of animal material-derived ingredients and are substantially free of clostripain, and thus are highly stable. “Substantially free” indicates that the collagenase contains less than 10 U clostripain per mg total collagenase, more preferably less than 5 U/mg, and most preferably about 1 U/mg or less, and/or that no visible band appears representing clostripain and/or degraded collagenase on SDS-PAGE gel compared to a reference standard.

Preferred methods for purifying collagenase involve using a “low glucose” medium as described herein, which contains less than about 5 g/L glucose, more preferably less than about 1 g/L, even more preferably less than about 0.5 g/L glucose, or is glucose-free, for culture of C. histolyticum. High salt concentrations in the growth media can reduce the amount of clostripain produced in culture, thus preferred media for C. histolyticum culture contain greater than about 5 g/L (or 0.5% w/v) total salt, more preferably greater than about 7.5 g/L (or 7.5%) total salt, and more preferably about 9 g/L (or 9%) or more. It is contemplated that any salt known to be suitable for use in microbiological fermentation media may be used. Chloride, phosphate or sulfate salts may be used. The salts may be sodium chloride, potassium chloride, monosodium phosphate, disodium phosphate, tribasic sodium phosphate, potassium monophosphate, potassium diphosphate, tripotassium phosphate, calcium chloride, magnesium sulfate or various combinations thereof. Potassium diphosphate may be about 0.1-0.3%, potassium phosphate may be about 0.75% to 0.175%, sodium phosphate may be about 0.2-0.5%, and/or sodium chloride may be about 0.15-0.35%. Preferably, the medium further comprises magnesium sulfate and vitamins, including, riboflavin, niacin, calcium pantothenate, pimelic acid, pyridoxine and thiamine.

Alternatively, the nutrient composition may contain 0.5-5% yeast extract, more preferably about 1-4%, and most preferably about 1.5-2.5%. Yeast extract is available from a variety of suppliers, including Cole Parmer (Vernon Hills, Ill.) and Fisher Scientific (Pittsburgh, Pa.).

The pH of the media is preferably between pH 7 and pH 8. Even more preferred is a pH between about pH 7.2 and about pH 7.7, most preferably about 7.4.

The collagenase contemplated for use with the invention can be any collagenase which is active under the necessary conditions. However, preferred compositions contain a mass ratio of collagenase I and collagenase II which is modified or optimized to produce a desired or even a maximal synergistic effect. Preferably, collagenase I and collagenase II are purified separately from the crude collagenase mixture produced in culture, and the collagenase I and collagenase II are recombined in an optimized fixed mass ratio. Preferred embodiments contain a collagenase Ito collagenase II mass ratio of about 0.5 to 1.5, more preferably 0.6 to 1.3, even more preferably 0.8 to 1.2, and most preferably, 1 to 1, however any combination or any single collagenase activity may be used.

A preferred method of producing collagenase which is contemplated for use with the invention involves fermenting C. histolyticum in a non-mammalian or non-animal medium, wherein the culture supernatant is substantially clostripain-free. The collagenases so produced can be isolated, purified, and combined to provide a composition for use in the invention which comprises a mixture of collagenase I and collagenase II in an optimized fixed mass ratio which is substantially clostripain-free. The crude collagenase obtained from fermentation of C. histolyticum may be purified by a variety of methods known to those skilled in the art, including dye ligand affinity chromatography, heparin affinity chromatography, ammonium sulfate precipitation, hydroxylapatite chromatography, size exclusion chromatography, ion exchange chromatography, and/or metal chelation chromatography. Additionally, purification methods for collagenases are known, such as, for example, those described in U.S. Pat. No. 7,811,560, which is incorporated herein in its entirety for all purposes.

Both collagenase I and collagenase II are metalloproteases and require tightly bound zinc and loosely bound calcium for their. Both collagenases have broad specificity toward all types of collagen. Collagenase I and Collagenase II digest collagen by hydrolyzing the triple-helical region of collagen under physiological conditions. Each collagenase shows different specificity (e.g. each have a different preferred target amino sequence for cleavage), and together they have synergistic activity toward collagen. Collagenase II has a higher activity towards all kinds of synthetic peptide substrates than collagenase I as reported for class II and class I collagenase in the literature.

The preferred collagenase consists of two microbial collagenases, referred to as Collagenase ABC I and Collagenase ABC II. The terms “Collagenase I”, “Aux I”, “ABC I”, and “collagenase ABC I” mean the same and can be used interchangeably. Similarly, the terms “Collagenase II”, “Aux II”, “ABC II”, and “collagenase ABC II” refer to the same enzyme and can also be used interchangeably. These collagenases are secreted by bacterial cells. Preferably, they are isolated and purified from Clostridium histolyticum culture supernatant by chromatographic methods. Both collagenases are special proteases and share the same EC number (E.C3.4.24.3). However, a collagenase or a combination of collagenases from other sources are contemplated for use with the invention. Collagenase ABC I has a single polypeptide chain consisting of approximately 1000 amino acids with a molecular weight of 115 kDa. Collagenase ABC II has also a single polypeptide chain consisting of about 1000 amino acids with a molecular weight of 110 kDa.

Collagenase acts by hydrolyzing the peptide bond between Gly-Pro-X, wherein X is often proline or hydroxyproline. Collagenase I acts at loci at ends of triple-helical domains, whereas Collagenase II cleaves internally. Hydrolysis continues over time until all bonds are cleaved.

Preferably, the collagenase product is at least 95% pure collagenase(s) and is substantially free of any contaminating proteases. More preferably, the collagenase product is 97% pure and most preferably 98% pure or more as determined by one or more of the following: sodium dodecyl sulfate polyacrylamide gel electrophoresis (SDS-PAGE); high performance liquid chromatography (HPLC); reverse-phase HPLC; or by enzymatic assays. The preferred collagenase product is essentially clostripain-free, and the purification preferably is performed in the absence of leupeptin. The preferred collagenase product for use with the invention has at least one specification selected from Table 1 below.

TABLE 1
Preferred Specifications for Collagenase Products

| | Specification | |
|---|---|---|
| Test | ABC-I | ABC-II |
| Appearance | Clear colorless and essentially free from | |
| | particulate matter | |
| Endotoxin | <10 EU/mL | |
| Identity (and purity) by | Major collagenase | Major collagenase |
| SDS-PAGE (Reduced | band between 98- | band between 97- |
| conditions, Coomasie) | 188 kDa | 200 kDa |
| | ≧95% | ≧95% |
| SRC assay (ABC-I) | 1967-3327 SRC | NA |
| | units/mg | |
| GPA assay (ABC-II) | | NA81934-119522 |
| | | GPA units/mg |

| | |
|---|---|
| Analysis of Proteins | >98% main peak; <2% aggregates by area |
| HPLC System | |
| (Aggregation by size | |
| exclusion | |
| chromatography) | |
| Identity and purity by | Major peak (ABC I or ABC II), ≧95% by |
| reverse phase liquid | area; Retention times of ABC-I and ABC-II |
| chromatography) | within 5% of reference |
| Clostripain assay (BAEE | ≦1 U/mg |
| assay) | |
| Bioburden | <1 cfu/mL |

The collagenase products described for use herein are useful for the treatment of collagen-mediated disease, including uterine fibroids, Dupuytren's disease; Peyronie's disease; frozen shoulder (adhesive capsulitis), keloids; tennis elbow (lateral epicondylitis); scarred tendon; glaucoma; herniated discs; adjunct to vitrectomy; hypertrophic scars; depressed scars such as those resulting from inflammatory acne; post-surgical adhesions; acne vulgaris; lipomas, and disfiguring conditions such as wrinkling, cellulite formation and neoplastic fibrosis.

In addition to its use in treating specific collagen-mediated diseases, the compositions of the invention also are useful for the dissociation of tissue into individual cells and cell clusters as is useful in a wide variety of laboratory, diagnostic and therapeutic applications. These applications involve the isolation of many types of cells for various uses, including microvascular endothelial cells for small diameter synthetic vascular graft seeding, hepatocytes for gene therapy, drug toxicology screening and extracorporeal liver assist devices, chondrocytes for cartilage regeneration, and islets of Langerhans for the treatment of insulin-dependent diabetes mellitus. Enzyme treatment works to fragment extracellular matrix proteins and proteins which maintain cell-to-cell contact. In general, the compositions of the present invention are useful for any application where the removal of cells or the modification of an extracellular matrix, are desired.

The collagenase compositions according to this invention are designed to administer to a patient in need thereof a therapeutically effective amount of a collagenase composition as described, or a therapeutically effective amount of a pharmaceutical collagenase formulation as described. A “therapeutically effective amount” of a compound, composition or formulation is an amount of the compound which confers a therapeutic effect on the treated subject, at a reasonable benefit/risk ratio applicable to any medical treatment.

The therapeutic effect may be objective (i.e., measurable by some test or marker) or subjective (i.e., subject gives an indication of or feels an effect), and may be determined by the clinician or by the patient. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, and diet of the patient; the time of administration, route of administration, and rate of excretion of the specific compound employed; the duration of the treatment; drugs used in combination or contemporaneously with the specific compound employed; and like factors well known in the medical arts.

The term “patient” or “patient in need” encompasses any mammal having a collagen-mediated disease or symptoms thereof. Such “patients” or “patients in need” include humans or any mammal, including farm animals such as horses and pigs, companion animals such as dogs and cats, and experimental animals such as mice, rats and rabbits.

Nanocarriers are designed to deliver and protect drug therapeutics (e.g. proteins, for example) from degradation. A nanocarrier formulation also is preferred because this method impedes diffusion and distribution of the drug away from the injected fibroid, prolongs release, delays inactivation, and therefore reduces the frequency of repeat injections. Any such nanocarrier known in the art can be used with the invention. Some of these nanocarriers also are referred to as thermoresponsive delivery systems.

Atrigel comprises a water-insoluble biodegradable polymer (e.g., poly(lactic-co-glycolic acid, PLGA) dissolved in a bio-compatible, water-miscible organic solvent (e.g., N-methyl-2-pyrrolidone, NMP). In use, collagenase is added to form a solution or suspension. Both the PLGA molecular weight and lactide-glycolide molar ratio (L:G ratio) governs drug delivery. Using an L:G ratio of from 50:50 to 85:15 and a polymer concentration of from 34 to 50%, clinical studies have demonstrated a depot which was maintained for more than 3 months.

ReGel® is a 4000 Da triblock copolymer formed from PLGA and polyethylene glycol (PEG, 1000 Da or 1450 Da) in repetitions of PLGA-PEG-PLGA or PEG-PLGA-PEG. ReGel® is formulated as a 23 wt % copolymer solution in aqueous media. A drug is added to the solution and upon temperature elevation to 37° C. the whole system gels. Degradation of ReGel® to final products of lactic acid, glycolic acid and PEG occurs over 1-6 weeks depending on copolymer molar composition. Chemically distinct drugs like porcine growth hormone and glucagon-like peptide-1 (GLP-1) may be incorporated, one at a time, and released from ReGel®.

LiquoGel™ can work by mechanistically independent drug delivery routes: entrapment and covalent linkage. Two or more drugs can be delivered to the tumor site using this carrier. LiquoGel™ is a tetrameric copolymer of thermogelling N-isopropylacrylamide; biodegrading macromer of poly(lactic acid) and 2-hydroxyethyl methacrylate; hydrophilic acrylic acid (to maintain solubility of decomposition products); and multi-functional hyperbranched polyglycerol to covalently attach drugs. LiquoGel™ generally is formulated as a 16.9 wt % copolymer solution in aqueous media. The solution gels under physiological conditions and degrades to release drug contents within 1-6 days.

Any of the above carriers can be used as a nanocarrier with the invention. A preferred nanocarrier, however, contains hyperbranched polyglycerols (HPG), which have many desirable features. HPGs grow by imperfect generations of branched units and are produced in a convenient single step reaction. Previous problems of large polydispersities in molecular weight in their production have been overcome. The resulting polymers contain a large number of modifiable surface functional groups as well as internal cavities for drug interaction. Other polymer approaches cannot easily provide these properties without significant increases in the number of synthetic steps and, consequently, cost. HPG polymers are based on glycerol and because of structural similarity with polyethylene glycol, is biocompatible.

Additional components optionally can be added to the polymer, therefore, modified HPG polymers and co-polymers of HPG are contemplated. These additional components or monomers can include, for example, crosslinks, biodegradable moieties, and thermoresponsive moieties. For example, thermally responsive hydrogels are attractive for injection therapy since it is possible to inject the necessary fluid volume from a syringe maintained below body temperature and upon warming, the mechanical properties are increased, thereby restraining the material at the injection site. Poly(N-isopropylacrylamide) (poly-NIPAAm) is a thermally responsive polymer with a lower critical solution temperature (LCST) of approximately 32° C. Copolymers of HPG with NIPAAm are therefore contemplated for use with the invention, and are preferred. This nanocarrier has a versatile mesh size and can be customized to entrap small drug molecules, large proteins, or a mixture of components, and gels at body temperature to permit slow release as the nanocarrier biodegrades.

In preferred embodiments of the invention, formulations exist as a liquid at temperatures below body temperature and as a gel at body temperature. The temperature at which a transition from liquid to gel occurs is sometimes referred to as the LCST, and it can be a small temperature range as opposed to a specific temperature. Materials possessing an LCST are referred to as LCST materials. Typical LCST's for the practice of the present invention range, for example, from 10 to 37° C. As a result, a formulation injected below the LCST warms within the body to a temperature that is at or above the LCST, thereby undergoing a transition from a liquid to a gel.

Suitable LCST materials for use with the invention include polyoxyethylene-polyoxypropylene (PEO-PPO) block copolymers. Two acceptable compounds are Pluronic acid F127 and F108, which are PEO-PPO block copolymers with molecular weights of 12,600 and 14,600, respectively. Each of these compounds is available from BASF (Mount Olive, N.J.). Pluronic acid F108 at 20-28% concentration concentration, in phosphate buffered Saline (PBS) is an example of a suitable LCST material. One beneficial preparation is 22.5% Pluronic acid F108 in PBS. A preparation of 22% Pluronic acid F108 in PBS has an LCST of 37° C. Pluronic acid F127 at 20-35% concentration in PBS is another example of a suitable LCST material. A preparation of 20% Pluronic acid F127 in PBS has an LCST of 37° C. Typical molecular weights are between 5,000 and 25,000, and, for the two specific compounds identified above are 12,600 and 14,600. More generally, materials, including other PEO-PPO block copolymers, which are biodisintegrable, and which exist as a gel at body temperature and as a liquid below body temperature can also be used according to the present invention. Further information regarding LCST materials can be found in U.S. Pat. Nos. 6,565,530 B2 and 6,544,227 B2.

Numerous thermosensitive hydrogels for the collagenase composition are known in the art and are commercially available. A preferred thermosensitive hydrogel for use in the formulation of the present invention is a triblock polymer of the structure PLGA-PEG-PLGA where PLGA represents poly (DL-lactic acid-co-glycolic acid) and PEG represents poly (ethylene glycol). A commercially available triblock polymer of these materials which has (PLGA:PEG:PLGA, LA/GA=3.1, PEG 1000-1500, Mn=3500-5500) is obtainable from Daigang Bio of Jinan, China as well as from Akina, Inc. of West Lafayette, Ind. 47906, USA. Another preferred thermosensitive hydrogel for use in the formulation of the present invention is poly(N-isopropylacrylamide) (poly-NIPAAm).

Pharmaceutical formulations of the collagenase compounds for the invention include a collagenase composition formulated together with one or more pharmaceutically acceptable vehicles or excipients. As used herein, the term “pharmaceutically acceptable carrier or excipient” means a non-toxic, inert, solid, semi-solid or liquid filler, diluent, encapsulating material, vehicle, solvent, or formulation auxiliary of any type, and may be made available in individual dosage forms or in bulk. Other dosage forms designed to create a depot of the active compound also are contemplated for use with the invention. Dosage forms for collagenase suitable for use with the invention include, but are not limited to lyophilized or other dried powder for reconstitution prior to injection, in multiple or single dose amounts, individual dosage units ready for injection (which preferably also include one or more preservatives), frozen unit dosage forms, or any mode of preparation known in the art. The formulations also may be provided in the form of a kit, which can contain the collagenase in solid form, liquid or solvent for reconstitution and injection, and any equipment necessary for administration, such as a syringe and needle, particularly a specialized syringe and/or needle for administration to an affected area. Preferably, the formulations are sterile. The products may be sterilized by any method known in the art, such as by filtration through a bacterial-retaining filter or are produced under aseptic conditions. Other methods include exposing the formulation or components thereof to heat, radiation or ethylene oxide gas.

Some examples of materials that can serve as pharmaceutically acceptable carriers are solvents for injection as known in the art. Examples include, but are not limited to sterile water, buffering solutions, saline solutions such as normal saline or Ringer's solution, pyrogen-free water, ethyl alcohol, non-toxic oils, and the like, or any solvent compatible with injection or other forms of administration as described herein for use with the invention.

In addition, any solid excipients known in the art for use in pharmaceutical products can be used with the invention as a vehicle or filler, for example. Sugars such as lactose, glucose and sucrose; starches such as corn starch and potato starch; cellulose and its derivatives such as microcrystalline cellulose, sodium carboxymethyl cellulose, ethyl cellulose and cellulose acetate; powdered tragacanth; malt; gelatin; gums; talc; glycols such as propylene glycol; esters such as ethyl oleate and ethyl laurate; agar, and the like can be used. Buffering agents compatible with the active compounds and the methods of use are contemplated for use, including acid or alkali compounds, such as magnesium hydroxide and aluminum hydroxide, citric acid, phosphate or carbonate salts and the like. Non-toxic compatible excipients such as lubricants, emulsifiers, wetting agents, suspending agents, binders, disintegrants, preservatives or antibacterial agents, antioxidants, sustained release excipients, coating agents and the like (e.g., sodium lauryl sulfate and magnesium stearate) also may be used, as well as coloring agents, perfuming agents, viscosity enhancing agents, bioadhesives, and the like, according to the judgment of the formulator.

For example, one or more biodisintegrable binders may be included in the formulations of the present invention, typically in connection with dosage forms having solid characteristics. Where employed, a wide range of biodisintegrable binder concentrations may be utilized, with the amounts varying based, for example, on the desired physical characteristics of the resulting dosage form and on the characteristics of the treatment agent that is selected (e.g., the degree of dilution, release delay, etc. that is desired/tolerated), among other considerations. The concentration of biodisintegrable binder typically ranges are from about 1 to 80 wt % of biodisintegrable binder, more typically about 5 to 50 wt %. A “biodisintegrable” material is one that, once placed in affected tissue, undergoes dissolution, degradation, resorption and/or other disintegration processes. Where such materials are included, formulations in accordance with the present invention will typically undergo at least a 10% reduction in weight after residing in tissue for a period of 7 days, more typically a 50-100% reduction in weight after residing in the tissue for a period of 4 days. Suitable biodisintegrable binders for use in connection with the present invention include, but are not limited to biodisintegrable organic compounds, such as glycerine, and biodisintegrable polymers, or any known disintegrant compound known in the art of pharmaceutics.

Where used, viscosity adjusting agent(s) are typically present in an amount effective to provide the formulation with the desired viscosity, for example, by rendering the formulation highly viscous, for example, in an amount effective to provide a viscosity between about 5,000 and 200,000 cps, more typically between about 10,000 and 100,000 cps, and even more typically between about 20,000 and 40,000 cps. By providing formulations having viscosities within these ranges, the formulations can be injected into tissue using conventional injection equipment (e.g., syringes). However, due to their elevated viscosities, the formulations have improved retention within the tissue at the injection site. The concentration of the viscosity adjusting agent(s) that is (are) used can vary widely. Commonly, the overall concentration of the viscosity adjusting agent(s) is between about 1 and 20 wt %. In many embodiments, the viscosity adjusting agents are polymers, which may be of natural or synthetic origin and are typically biodisintegrable. The polymers are also typically water soluble and/or hydrophilic. However, in some embodiments, for instance where an organic solvent such as dimethylsulfoxide (DMSO) is used as a liquid component, the viscosity adjusting agent can be relatively hydrophobic. The polymeric viscosity adjusting agents include homopolymers, copolymers and polymer blends.

Examples of viscosity adjusting agents for the practice of the present invention include, but are not limited to the following: cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, hydroxyethyl starch (HES), dextran, dextran derivatives, chitosan, and alginic acid and its various salts, carrageenan, various gums, including xanthan gum, guar gum, gum arabic, gum karaya, gum ghatti, konjac and gum tragacanth, glycosaminoglycans and proteoglycans such as hyaluronic acid and its salts, heparin, heparin sulfate, dermatan sulfate, proteins such as gelatin, collagen, albumin, and fibrin, other polymers, for example, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide and poly(ethylene oxide-propylene oxide) (e.g., Pluronic acid), polyoxyethylene (polyethylene glycol), polyethyleneamine and polypyrridine, poly-metaphosphate (Kurrol salts), polyvinyl alcohol, additional salts and copolymers beyond those specifically set forth above, and blends of the foregoing (including mixtures of polymers containing the same monomers, but having different molecular weights), and so forth. Many of these species are also useful as binders.

In other embodiments of the invention, formulations or carriers are crosslinked, either prior to use or in vivo. Crosslinking is advantageous, for example, in that it acts to improve formulation retention (e.g., by providing a more rigid/viscous material and/or by rendering the polymer less soluble in a particular environment). Where the formulation is crosslinked in vivo, a crosslinking agent is commonly injected into tissue either before or after the injection or insertion of a formulation in accordance with the present invention. Depending on the nature of the formulation and the crosslinking agent, the formulation may be converted, for example, into a solid, into a semi-solid, or into a high-viscosity fluid.

Crosslinking agents suitable for use in the present invention include, any non-toxic crosslinking agent, including ionic and covalent crosslinking agents. For example, in some embodiments, polymers are included within the formulations of the present invention, which are ionically crosslinked, for instance, with polyvalent metal ions. Suitable crosslinking ions include polyvalent cations selected from the group consisting of calcium, magnesium, barium, strontium, boron, beryllium, aluminum, iron, copper, cobalt, lead and silver cations ions. Polyvalent anions include phosphate, citrate, borate, succinate, maleate, adipate and oxalate anions. More broadly, crosslinking anions are commonly derived from polybasic organic or inorganic acids. Ionic crosslinking may be carried out by methods known in the art, for example, by contacting ionically crosslinkable polymers with an aqueous solution containing dissolved ions.

In some embodiments, polymers are included, which are covalently crosslinkable, for example, using a polyfunctional crosslinking agent that is reactive with functional groups in the polymer structure. The polyfunctional crosslinking agent can be any compound having at least two functional groups that react with functional groups in the polymer. Various polymers described herein can be both covalently and ionically crosslinked.

Suitable polymers for ionic and/or covalent crosslinking can be selected, for example, from the non-limiting list of the following: polyacrylates; poly(acrylic acid); poly(methacrylic acid); polyacrylamides; poly(N-alkylacrylamides); polyalkylene oxides; poly(ethylene oxide); polypropylene oxide); poly(vinyl alcohol); poly(vinyl aromatics); poly(vinylpyrrolidone); poly(ethylene imine); poly(ethylene amine); polyacrylonitrile; poly(vinyl sulfonic acid); polyamides; poly(L-lysine); hydrophilic polyurethanes; maleic anhydride polymers; proteins; collagen; cellulosic polymers; methyl cellulose; carboxymethyl cellulose; dextran; carboxymethyl dextran; modified dextran; alginates; alginic acid; pectinic acid; hyaluronic acid; chitin; pullulan; gelatin; gellan; xanthan; carboxymethyl starch; hydroxyethyl starch; chondroitin sulfate; guar; starch; and salts, copolymers, mixtures and derivatives thereof

Preferred collagenase compositions for use in the invention comprise a mixture of collagenase I and collagenase II has a specific activity of at least about 700 SRC units/mg, such as at least about 1000 SRC units/mg, more preferably at least about 1500 SRC units/mg. One SRC unit will solubilize rat tail collagen into ninhydrin reaction material equivalent to 1 nanomole of leucine per minute, at 25° C., pH 7.4. Collagenase has been described in ABC units as well. This potency assay of collagenase is based on the digestion of undenatured collagen (from bovine tendon) at pH 7.2 and 37° C. for 20-24 hours. The number of peptide bonds cleaved are measured by reaction with ninhydrin. Amino groups released by a trypsin digestion control are subtracted. One net ABC unit of collagenase will solubilize ninhydrin reactive material equivalent to 1.09 nanomoles of leucine per minute. One SRC unit equal approximate 6.3 ABC unit or 18.5 GPA unit. In one embodiment, each milligram of collagenase for injection will contain approximately 2800 SRC units.

Doses contemplated for administration by direct injection to the affected tissue will vary depending on the size of the tissue to be treated and the discretion of the treating physician. However, doses generally are about 0.06 mg collagenase to about 1 mg collagenase per cm³ of tissue to be treated or about 0.1 mg collagenase to about 0.8 mg collagenase per cm³ of tissue to be treated, or about 0.2 mg collagenase to about 0.6 mg collagenase per cm³ of tissue to be treated.

Formulations that contain an additional active agent or medication also are contemplated. Optional additional agents which can be included in the formulation for concomitant, simultaneous or separate administration include, for example, any pharmaceutical known in the art for shrinkage, treatment or elimination of the collagen-mediated diseases or their symptoms, or to assist in performance of the present treatment methods. For example, one or more fibroid treatment agents such as aromatase inhibitors (e.g., letrozole, anastrozole, and exemestande), progesterone receptor agonists and modulators (e.g., progesterone, progestins, mifepristone, levonoergestrel, norgestrel, asoprisnil, ulipristal and ulipristal acetate, telepristone), selective estrogen receptor modulators (SERMs) (e.g., benzopyran, benzothiophenes, chromane, indoles, naphthalenes, tri-phenylethylene compounds, arzoxifene, EM-652, CP 336,156, raloxifene, 4-hydroxytamoxifen and tamoxifen), gonadotrophin-releasing hormone analogs (GnRHa) (e.g., GnRH agonist peptides or analogs with D-amino acid alterations in position 6 and/or ethyl-amide substitutions for carboxyl-terminal Gly10-amide such as triptorelin or GnRH antagonists such as cetrorelix, ganirelix, degarelix and ozarelix), growth factor modulators (e.g., TGFb neutralizing antibodies), leuprolide acetate, non-steroidal anti-inflammatory drugs, inhibitors of the mTOR pathway, inhibitors of the WNT signaling pathway, vitamin D, vitamin D metabolites, vitamin D modulators, and/or an additional anti-fibrotic compound (e.g., pirfenidone and halofuginone) may be co-administered with collagenase in the same or a separate administration.

Chemical ablation agents also can be included in the formulations of the present invention. In effective amounts, such compounds cause tissue necrosis or shrinkage upon exposure. Any known ablation agent can be used according to the art, in concentrations as appropriate to the conditions while avoiding inactivation of the collagenase, with the amounts employed being readily determined by those of ordinary skill in the art. Typical concentration ranges are from about 1 to 95 wt % of ablation agent, more typically about 5 to 80 wt %. Ablation agents suitable for use with the invention include, but are not limited to osmotic-stress-generating agents (e.g., a salt, such as sodium chloride or potassium chloride), organic compounds (e.g., ethanol), basic agents (e.g., sodium hydroxide and potassium hydroxide), acidic agents (e.g., acetic acid and formic acid), enzymes (e.g., hyaluronidase, pronase, and papain), free-radical generating agents (e.g., hydrogen peroxide and potassium peroxide), oxidizing agents (e.g., sodium hypochlorite, hydrogen peroxide and potassium peroxide), tissue fixing agents (e.g., formaldehyde, acetaldehyde or glutaraldehyde), and/or coagulants (e.g., gengpin). These agents may be combined with collagenase in the same formulation so long as they do not negatively affect the enzymatic activity of the collagenase, or they may be administered separately, at the same time or at different times.

The methods according to the invention may be used in conjunction with any known treatments to control symptoms caused by collagen-mediated diseases, for example, NSAIDs, and other analgesics can be used to reduce pain.

In a preferred embodiment of the present invention thermosensitive hydrogel materials known in the art which do not meet the requirements of injectability or compatibility due to viscosity or acidic pH can be treated in solution to modify their properties by adding to their solutions a viscosity adjusting or pH adjusting amount of the compound tris (hydroxymethyl) amino methane. In this manner such hydrogel properties will be modified to allow injection through a 28 G½ needle without jamming and at a neutral or slightly basic pH, will be compatible with collagenase.

A suitable collagenase formulation for non-clinical testing can be prepared by dissolving 1 mg collagenase and 1.7 mg of a polysaccharide carrier material such as lactose in 0.5 ml of 13%-15% triblock hydrogel solution, such as PLGA-PEG-PLGA with pH adjusted to a pH 8.5 by the addition of tris (hydroxymethyl) amino methane. Such resulting solution can be readily introduced into a insulin syringe through a 28 G½ needle. The basic pH has been found to be a key to having an acceptable injectability. Collagenase has been found to be stable when maintained in gels formed from the recipe when held at 37° C. for at least 48 hours. Additionally, it has been found that released and entrapped collagenase from such gels have the same biological activity as the untreated collagenase. In certain embodiments where the hydrogel exhibits a sensitivity to basic conditions it is preferred that the tris (hydroxymethyl) amino methane can be added to the hydrogel solution just prior to mixing with the collagenase powder in order to minimize any risk of degradation.

In order to provide a formulation suitable for injection the hydrogel solution has to be sterilized. Any method not involving elevated temperatures or use of materials which might affect the integrity of the hydrogel may be employed. A preferred sterilization method involves filtering the hydrogel solution through a small pore filter such as, for example, a filter with pores of about 0.22 μm into a sterile, sealable container. The resulting sterile solution can be conveniently stored prior to use as a frozen stock solution. This stock solution can be thawed when needed and used as diluent to dissolve lyophilized collagenase provided before injection.

In a further embodiment of the invention the needed components for effecting treatment of a subject for a target indication can be conveniently provided to the medical professional in kit form. Such kit would contain a sterile vial containing the thermosensitive hydrogel stock solution in an amount sufficient to provide one or more injections, one or more vials each containing a therapeutic dose for the target indication of collagenase as a lyophilized powder and optionally a package insert approved by the drug regulatory authority in the jurisdiction where the kit is to be used in treating a patient. In embodiments where the hydrogel Is sensitive to extended exposure to base conditions, It Is preferable to provide the tris (hydroxymethyl) amino methane solution In a separate vial. Most preferably the vials will be store at refrigerator or frozen conditions before use.

The preparation and use of formulations of the present invention are further illustrated by reference to the Examples which follow. It should be understood that the scope and nature of the present invention are to be defined by the claims of this application and should be not limited in any way by such Examples.

### EXAMPLE 1

### PLGA-PEG-PLGA-Collagenase Polymer Solution: Preparation and Characterization

Preparation of Polymer Stock Solution

A triblock polymer, poly (DL-lactic acid-co-glycolic acid)-poly (ethylene glycol)-poly(DL-lactic acid-co-glycolic acid), (PLGA-PEG-PLGA) (Mn=1600-1500-1600) was obtained from Daigang Bio., Jinan China. A 15% (w/v) polymer solution was prepared by mixing dry polymer and water at 2-8° C. The dissolution may take a few days under gentle agitation. The solution was then filtered through a 0.22 μm filter. The sterilized solution is aliquoted and stored at-20° C. The frozen solution is preferably placed at refrigerator temperature overnight prior to preparing the collagenase-hydrogel solution.

A hyperbranched polyglycerols (HPG) polymer, poly(NIPAAm-co-HEMAPLA-co-AAc-co-HPG-MA)copolymers with a ratio of (83/7/1/9) was made by NCCU. A 20% (w/v) polymer solution is prepared by mixing dry polymer and water at 2-8° C. The dissolution may take a few days under gentle agitation. The solution is then filtered with 0.22 μm filter. The sterilized solution can be aliquot and stored at −20° C. The frozen solution is preferably placed at refrigerator for overnight prior to making collagenase-hydrogel solution.

Method of Polymer Dilution

The polymer solution is further diluted to 13% with water for PLGA-PEG-PLGA and 17% for HPG polymer, poly(NIPAAm-co-HEMAPLA-co-AAc-co-HPG-MA)copolymer. This solution has a pH of 4. The solution is capable of forming a soft gel at 37° C. In addition to the acidic condition, which causes collagenase denaturing, the 13% or 17% polymer solution was also found to be viscous at room temperature.

Many published results are in fact from using chilled polymer solution, normally 4° C. A temperature of 4° C. is less than ideal as a clinical working condition, which normally prefers an ambient temperature. This viscosity makes it impossible to use in a syringe. Tris buffer of pH 7.5 was then used to dilute the polymer solution. The collagenase is now safe, but the polymer solution was still too thick to be handled in a syringe at room temperature. Adjusting to pH 8.5 was found to substantially reduce the viscosity of the polymer solution at room temperature. The pH 8.5 polymer solution was a clear, fluidic solution and can be handled by a syringe with a 28 G½ needle.

Preparation of Collagenase/Hydrogel Solution

Collagenase/hydrogel solution may is prepared as follows: (A) add a calculated volume of sterile 0.75 M tris buffer, pH 8.5 into a sterile polymer solution (Example 1); and (B) add a required volume of polymer solution to lyophilized collagenase powder. The final concentration of the polymer is 13% (w/v) for PLGA-PEG-PLGA and 17% (w/v) for HPG polymer, poly(NIPAAm-co-HEMAPLA-co-AAc-co-HPG-MA)copolymer. The dissolved collagenase is preferably left in a refrigerator for 30 minutes prior to injection for clearing up the bubbles.

### EXAMPLE 2

### Syringe Test at Room Temperature-Needle Test at Body Temperature

Many thermosensitive hydrogel solutions are viscous and pose a challenge for use in a syringe at room temperature: withdrawing, expelling air etc. especially when a small size of syringe and needle is needed. A syringe test is performed using a small size of syringe and 28 G½ needle. An acceptable polymer solution should be easily handled with a small size of syringe and 28 G½ needle at room temperature. The current mode of injection of collagenase solution is by intra-lesion injection, which often requires a clinician to spend time doing needle placement before pushing the plunger. Since the needle has already entered the body, gelation may occur prior to discharging the contents of the syringe. A needle test is performed by immersing the needle into buffer warmed to 37° C. for up to 40 seconds before pushing the plunger to release the hydrogel solution.

The syringe tests demonstrate that collagenase-hydrogel solution (0.25 mL) can be handled like collagenase-saline solution. The needle tests show that the collagenase/hydrogel can be discharged easily at body temperature.

### EXAMPLE 3

### Sterilization Method

Polymer solutions can be sterilized by filtration at 4° C. through a 0.22 μm filter.

### EXAMPLE 4

### Compatibility, Initial Entrapment and Collagenase Release Test with SRC Assay

Collagenase activity can be measured by a biological potency assay method—the SRC assay. This method uses soluble rat trail tendon collagen as a substrate. The assay is based on the method originally developed by Mallya (Mallya, S. K., et al. (1986) Anal. Biochem. 158: 334-345). The collagenase activity is measured by the amount of degraded collagen, (small peptide fragments) which is quantified by the Ninhydrin reaction. The optical density of the reaction solution (purple Ninhydrin) is measured with a spectrometer at 570 nm and compared with the ninhydrin reaction using a known amount of leucine (standard curve). The nmol peptide hydrolyzed is calculated into nmol leucine equivalent. The unit of collagenase activity was expressed as nmol leu equiv./min.

A 200 μl of collagenase/hydrogel solution was placed into a test tube with 1 mL Tris buffer (20 nM Tris(hydroxymethyl)amino methane/4 mM calcium acetate pH 7.4) pre-warmed at 37° C. The gelation occurred instantly. The test tubes were incubated for various times up to 48 hr. The collagenase potency of supernatant and gel were measured with the SRC assay. The result in Table I indicate that the collagenase is compatible with the polymer and gelation process. The results show that initially more than 80% of the collagenase is entrapped in the gel. The results also show that most of the collagenase is released from the gel in 48 hours. A SDS-PAGE test showed a similar entrapment rate and release pattern. In contrast to most slow release hydrogels, the release for the present formulation is much faster. This relative “fast” slow release is more desirable for clinical uses.

TABLE I

| | 1 hr. | 24 hr. | 48 hr. |
|---|---|---|---|
| Collagenase in test tube | 100% | 93% | 98.1% |
| Collagenase in supernatant | 13% | 51.7% | 80% |

### EXAMPLE 5

### Compatibility Test with GPA Assay

The hydrogel's compatibility is also verified with the second biological potency assay—GPA assay, a synthetic peptide substrate based assay. Carbobenzoxy-glycyl-L-prolyl-glycyl-glycyl-L-prolyl-L-alanine (zGPGGPA) is a synthetic substrate for Clostridial collagenase. This substrate is readily cleaved by Aux II collagenases (collagenase ABC II) into the two peptides; carbobenzolxy-glycyl-L-prolyl-glycine (zGPG) and glycyl-L-prolyl-L-alanine (GPA). The released free amino group on GPA is reacted with ninhydrin reagent. The optical density of purple ninhydrin reaction solution is measured with a spectrometer at 570 nm and compared with the ninhydrin reaction from to collagenase reference standard. The unit of collagenase activity was expressed as nmol leu equiv./min. This assay procedure was originally developed by W. Appel [in H. U. Bergmeyer, ed., Methods of Enzymatic Analysis; New York: Academic Press/Verlag Chemie, 1974].

A total of 0.353 mg of collagenase was mixed with 0.3 mL 13.2% triblock hydrogel solution, pH 8.5. 0.2 mL collagenase hydrogel solution was added to 1 mL 37° C. tris buffer in a test tube. The gelation occurred instantly. The test tube was placed on a rocker for 1 hr. at 37° C. The collagenases which went through the gelation process was compared with a control collagenase using the GPA assay. The results of 51473 units/mg for the control collagenases and 51182 units/mg for the collagenase in the gel indicate that the collagenases were compatible with the polymer.

1. A composition for treating a collagen-mediated disease, comprising collagenase and a carrier that provides sustained release of an amount of said collagenase sufficient to treat said collagen-mediated disease.

2. The composition of claim 1, wherein said carrier comprises gel-forming polymers.

3. The composition of claim 2, wherein said polymer is a triblock polymer or a copolymer based on N-siopropylacrylamide (NIPAAm)

4. The composition of claim 3, wherein said triblock polymers comprise poly(lactic-co-glycolic acid) (PLGA) and polyethylene glycol (PEG).

5. The composition of claim 4, wherein said polymers comprise a copolymer formed from PLGA and polyethylene glycol (PEG).

6. The composition of claim 5, wherein the PLGA and PEG copolymers are formed in repetitions of PLGA-PEG-PLGA or PEG-PLGA-PEG.

7. The composition of claim 1, wherein said composition is injectable, insertable or applied topically.

8. The composition of claim 1, which can be administered through a syringe fitted with a 28 G½ needle without pre-gelation in the needle on injection.

9. The composition of claim 8, wherein syringeability or compatibility has been modified by the addition to the formulation of tris (hydroxymethyl) amino methane.

10. The composition of claim 9, comprising tris (hydroxymethyl) amino methane in an amount sufficient to provide a pH of about 8.5.

11. The composition of claim 3, wherein the NIPAAm based polymer is a copolymer based on N-siopropylacrylamide (NIPAAm) and one or more of polyactide-hydroxyethyl methacrylate (HEMAPLA), acrylic acid (AAc), and methacrylated hyperbranched polyglycerol (HPG-MA).

12. The composition of claim 11, comprising copolymers of poly-NIPAAm and hyperbranched polyglycerols (HPG).

13. The composition of claim 3, having a lower critical solution temperature (LCST) of 10-37° C.

14. The composition of claim 13, having a LCST of approximately 25-32° C.

15. The composition of claim 1, wherein the composition exists as a liquid at temperatures below body temperature and as a gel at body temperature.

16. The composition of claim 1, wherein the collagenase is obtained from Clostridium histolyticum.

17. The composition of claim 1, wherein the collagenase is a mixture of collagenase I and collagenase II.

18. A method for treating a subject suffering from a collagen-mediated disease, comprising administering to said subject the composition of claim 1 in an amount sufficient to treat said collagen-mediated disease.

19. The method of claim 18, wherein said disease is selected from Dupuytren's contracture, Peyronie's disease, frozen shoulder, human lipoma, canine lipoma, cellulite, uterine fibroids, chronic dermal ulcers and severely burned areas.

20. A kit for providing at least one therapeutic dose of a formulation of claim 1, said kit comprising in a unit package at least one container containing a sterile thermosensitive hydrogel fluid in an amount sufficient for at least one therapeutic dose; at least one second container containing an effective amount of collagenase in lyophilized, powder form: and a package insert.
